(19) United States
(12) Patent Application Publication
(10) Pub. No.: US 2003/0018179 A1
(43) Pub. Date: Jan. 23, 2003

(54) Method for generating nucleation centers for selective heterogeneous growth of metal clusters on DNA molecules

(75) Inventors: Michael Mertig, Dresden (DE); Lucio Colombi-Ciacchi, Dresden (DE); Ralf Seidel, Dresden (DE); Wolfgang Pompe, Hartha (DE); Alessandro De Vita, Trieste (IT)

Correspondence Address: Gudrun E. Huckett, P.O. Box 3187, Albuquerque, NM, 87190-3187, US

(73) Assignee: Technische Universitat Dresden, Dresden (DE)

(21) Appl. No.: 10/177,775

(22) Filed: Jun. 21, 2002

(30) Foreign Application Priority Data
Jun. 22, 2001 (DE) 101 31 651
Jun. 19, 2002 (DE) 102 28 056

(51) Int. Cl. C07H 21/04

(52) U.S. Cl. 536/23.1

(57) ABSTRACT

The invention relates to a novel method for generating nucleation centers for the selective heterogeneous metal cluster growth on DNA molecules by employing metal complexes which are being bonded covalently to the bases of the DNA. The nucleation centers serve as preferred locations of chemical deposition of metals on the DNA from a solution so that clusters or thin metal films can be grown on the biological template without metallic deposition products being formed homogeneously in the solution. The method provides the possibility of controlling the metallization via the sequence of the employed DNA and is thus in principle sequence-specific.

The invention relates to a novel method for generating nucleation centers for the selective heterogeneous growth of metal clusters on DNA molecules by using metal complexes which are being bonded covalently to the bases of the DNA. The nucleation centers serve as preferred locations of chemical deposition of metals on the DNA from a solution by which method clusters or thin metal films can be grown on the biological template without metallic deposition products forming homogeneously in the solution. The method provides the possibility of controlling the metallization via the sequence of the employed DNA and is thus, in principle, sequence-specific. The invention can be employed for manufacturing metallic nanostructures on the basis of DNA which enable, for example, applications in nano-electronic devices, by metallization of individual DNA molecules, or assembled networks in solution, or DNA immobilized on surfaces.

DNA as a carrier molecule of the genetic information provides by way of the Watson-Crick base pair formation the possibility of self assembly and provides thus also the possibility of building structures whose formation can be controlled artificially via the base sequence [(N. C. Seeman, Trends in Biotechnology 17, 437 (1999)]. This special capability and the possibility of association with specific proteins focus interest on the unique macromolecule DNA increasingly also in the field of engineering. The main goal in this connection is a combination of the assembly capability of the DNA with the generation of large nanostructures with novel optical or electronic properties.

For arranging inorganic nanoparticles on the DNA it is known to bind finished colloidal substances, for example, via electrostatic interaction, on the DNA [T. Torimoto et al., J. Phys. Chem. B 103, 8800 (1999)]. In this connection, the biomolecule functions only as a support along which the colloidal substances will be arranged. This ordered arrangement can be easily canceled, however, when the chemical environment changes, for example, by a change of the pH value; this is a disadvantage for technical applications.

From WO 97/48837 A1 metallic nanostructures on the basis of self-assembling geometrically highly ordered proteins are known. The assembled proteins are activated by a metal salt and subsequently metallized without current in a metallization bath under conditions which are compatible with proteins. For a partial metallization only the activation with a metal solution and the active participation of the amino acid groups forming the proteins structure are employed for the reduction of the metal complex.

As a result of the activation small noble metal or heavy metal particles which are visible by electron microscopy are produced which, when applying a suitable metallization bath, serve as metallic nucleation centers for a further catalytic metal deposition. These metal particles result because the buffer solution employed in WO 97748837 A1 intrinsically has a weak reducing effect on the metal solution for employed for activation. This means that in the activation process described therein only one chemical reduction is involved which results in the formation of metallic nucleation centers in the form of metal particles.

A disadvantage is that this method does not utilize the specificity of the DNA base sequence for a nucleotide-controlled metallization because the formation of small Pt clusters does not take place with preference on a particular base or a special base pattern in the incubation phase but on uncontrollable locations of the molecule. Moreover, the homogenous nucleation of metal clusters in the solution is not excluded by the method described in WO 97/48837 A1. This results in aggregation of homogeneously formed particles which cause, in the end, a relatively coarse metallization of the DNA templates.

It is an object of the invention to provide a method for selective heterogeneous metallization of DNA which directly employs the sequence-specific properties of DNA.

According to the invention, this object is solved by a method for providing specific nucleation centers for the heterogeneous cluster growth on DNA. In this connection, a DNA solution is initially incubated together with a metal salt solution and, subsequently, the entire solution which contains metal complex-DNA-adducts as well as free metal complexes is reduced by means of suitable reducing agents. In contrast to the method described in WO 97/48837 A1, during the incubation phase no reducing agent is used so that in the incubation phase no metallic particles are generated.

The method is expandable in the sense that the DNA solution for the reduction can be dialyzed so that metal salt complexes which are not bonded to the DNA can be partially removed from the solution and, in this way, the metal salt quantity converted in sum total to metal can be controlled.

Surprisingly, it was found that the complexes bonded to DNA act very efficiently as nucleation centers for the further metal deposition. This has the effect that by means of the formation of the nucleation centers via the incubation of the DNA with dissolved metal salts the balance between homogenous cluster nucleation in solution and heterogeneous nucleation of clusters on DNA with increasing incubation time can be pushed in the direction toward the heterogeneous nucleation. Accordingly, the metal deposition on the DNA molecules becomes controllable. Under advantageous conditions (e.g., see Example 1) the heterogeneous nucleation of metal clusters on the DNA is so dominating that the homogenous nucleation of clusters in solution is suppressed completely even though in the metallization bath a great excess of metal salt ions not bonded to DNA is present. In this way, a DNA with metallic nucleation centers can act as a molecular promoter for the heterogeneous nucleation of auto-catalytically growing metal clusters.

In an advantageous embodiment of the method according to the invention a DNA solution is incubated with a solution of a bi-valent platinum salt at room temperature and, subsequently, the solution comprised of Pt(II)-DNA-adducts and dissolved Pt(II) complexes is reduced by means of suitable reducing agents.

The chemical bonding of noble metal complexes on DNA is very well elucidated for certain groups of metals, in particular, for cis-platin which has been employed broadly for many years as a result of its anti-mitotic effect in chemotherapy of cancer diseases (B. Lippert, (Ed.), Cis-platin: chemistry and biochemistry of a leading anti-cancer drug, Wiley-VCH, Weinheim, 1999). Cis-platin bonds sequence-specifically to the bases of the DNA. The preferred binding location is the so-called N7 position of guanine wherein first a mono-functional and subsequently a bi-functional bonding of hydrolyzed complexes can be observed [D. P. Bancroft, C. A. Lepre, S. J. Lippard, J. Am. Chem. Soc. 112, 6860 (1990); J. -P. Macquet, T. Theophanides, Biopolymers 14, 781 (1975)]. Tetrachloro platin shows a similar coordination behavior (E. T. Sacharenko, Yu. S. Moschkowski, Biophysica, 7, 373 (1972), in Russian).

According to the invention, the metal ions after completed covalent bonding on specific bases of the DNA act as particularly effective centers for heterogeneous nucleation of metal clusters in the reduction processes. This result could not be expected. Experiments undertaken by the inventors have shown that the strong electron donor character of the nucleotides is responsible for this so that the generated Pt-Pt bonds are particularly stabilized.

According to the invention, the preferred coordination of Pt complexes on guanine enables a sequence-specific metallization of DNA because metal is deposited only where prior nucleation centers have been generated on the DNA template molecule. In this way, via the sequence of the bases or specific base patterns a location-specific metallization of the DNA can be realized wherein defined parts of the molecule can be metallized and others cannot be metallized.

Advantageous for the metallization of DNA according to the invention is that after completed generation of specific nucleation centers by means of the chemical bonding of one type of metal complexes on the base pairs it is indeed possible to grow metal of another type in the subsequent chemical reduction. According to the invention, the chemical reduction is a single-step method.

With respect to the chemical reduction, the described method can advantageously be expanded to a multi-step method wherein each employed metal type can be chemically reduced in a separate step. In this connection, the metal nuclei which are produced in the first process step on the DNA provide the nucleation centers for the subsequent deposition reaction.

According to the invention, the cluster growing on the DNA do not require further ligands or so-called “capping agents” for their stabilization. The DNA acts as a protective polymer, which prevents agglomeration of the metal clusters. However, the addition of further protective ligands results in an additional stabilization of the Pt cluster on the DNA. As a result of this, a significantly enlarged cluster chain length can be observed.

With the aid of the attached embodiments the invention will be explained in more detail:

### EXAMPLE 1

Manufacture of Regular Chains of Platinum Clusters on λ DNA

A solution of 5 μg/ml of λ-DNA (New England Biolabs, U.S.A.) is incubated for approximately 20 hours with an aged 1 mM solution K₂PtCl₄ (Fluka, Buchs, Switzerland) at room temperature wherein a complex to nucleotide ratio of approximately 65:1 is maintained. During the incubation a part of the Pt complex covalently bonds on the bases of the employed DNA. After the incubation time, 10 mM dimethyl amino borane (DMAB, Fluka) is added to the DNA metal salt solution at a temperature of 3° C. in stoichiometric excess relative to the Pt complexes in order to reduce the Pt complexes to metallic platinum (Pt(0)). The reaction kinetics is monitored by means of UV-VIS spectroscopy. The reaction products are examined by means of atomic force microscopy or electron microscopy.

If DNA or other stabilizing agents are not present, small metal clusters are formed during the process of reduction which agglomerate quickly to agglomerates of a typical diameter of approximately 50-100 nm. They are also formed in the presence of DNA for very short incubation times. The cluster agglomeration is increasingly suppressed with growing DNA concentration. With increasing incubation time, heterogeneous nucleation increases and the preferred growth of clusters on the DNA thus results. For long incubation times (approximately 20 hours), a complete suppression of the homogenous nucleation of clusters in solution is observed.

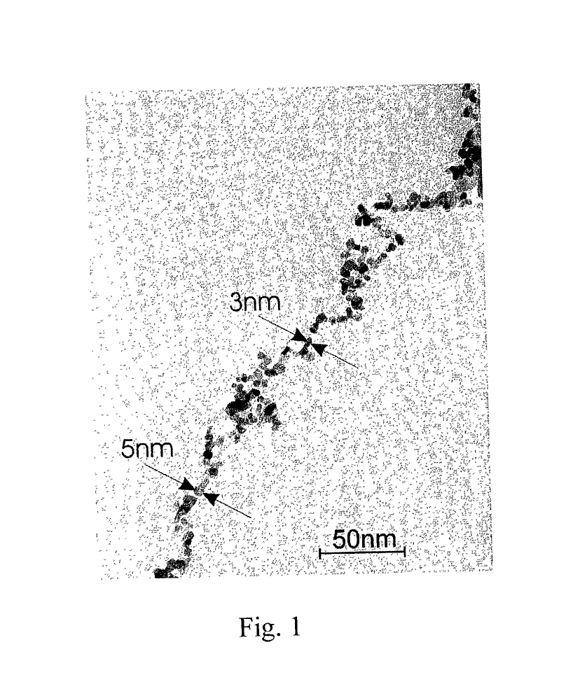

FIG. 1 shows a TEM image (160 kV; magnification rate:275,000) of a Pt cluster chain which has been grown by means of the described method along a DNA strand. Cluster chains generated in solutions have been deposited on a thin carbon film in order to enable microscopic examination. All metal clusters are arranged along the DNA molecules. The selective heterogeneous growth of cluster chains on the molecules is observed wherein the clusters have an average diameter of approximately 4 nm.

### EXAMPLE 2

Sequence-Specific Metallization of DNA

A metallization of three different DNA types is carried out according to the chemical procedure described in Example1. In this context, DNA of Clostridium perfringens with (GC) base pair contents of 26.5%, salmon sperm DNA (Salmon testes) with a GC contents of 41.2%, and DNA of Micrococcus lysodeikticus with a GC contents of 72% were used (all types can be ordered from SIGMA-ALDRICH).

By means of UV-VIS spectroscopy the reaction kinetics for the different DNA types and for different incubation durations with the platinum salt were examined. It was observed that the reaction kinetics depended greatly on the different DNA types. For small and average incubation durations the reaction kinetics is accelerated with increasing GC contents. For very long incubation durations there are only minimal differences with respect to the reaction kinetics.

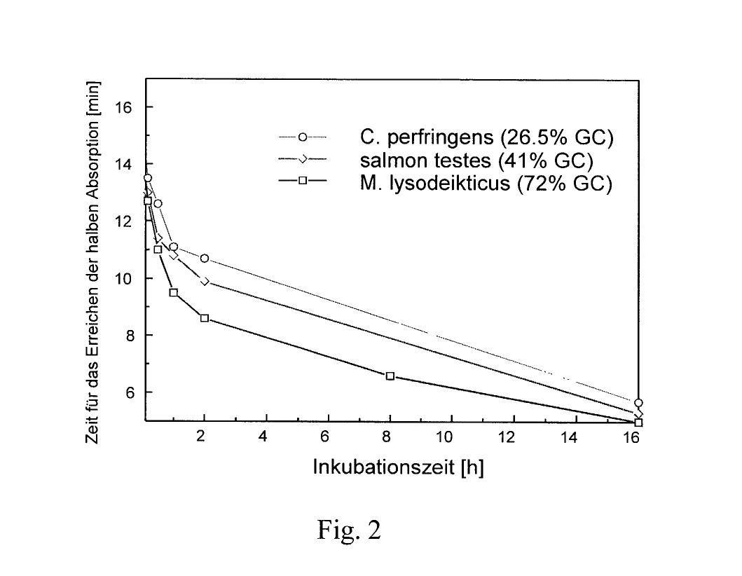

FIG. 2 shows the results for the measured kinetics of the cluster growth on the DNA as a function of the GC base pair contents of the DNA. The x axis indicates the duration of incubation of DNA with the Pt complexes. During the course of incubation the DNA is covered more and more densely with complexes. The y axis is a measure for the time in which the reduction reaction takes place subsequent to incubation. It was determined by means of optical absorption. The more complexes are bonded on the DNA, the faster the cluster growth takes places, i.e., the smaller the time at which half the final absorption is reached. For DNA with high GC contents more complexes are bonded per time unit than in the case of DNA with a low GC contents. This also shows that the cluster growth for DNA with high GC contents occurs within a shorter time period than in the case of DNA with a low GC contents.

The observed behavior coincides with the bonding behavior of complexes on DNA and thus establishes direct proof that the generation of nucleation centers for the heterogeneous cluster formation takes place during the reduction by means of bonding of metal complexes on the DNA bases during incubation. At the same time, by means of this mechanism which is the basis of the invention as sequence-controlled, location-specific metallization of DNA can be realized.

### EXAMPLE 3

Cluster Nucleation and Cluster Growth with Different Metal Complexes in One Reduction Step

The procedure described in Example 1 can be performed also with other metal salts. In addition to tetrachloro platin, cis platin can also be used for generating nucleation centers. Moreover, for the growth of the clusters different metal salts can be used.

For this purpose, after incubation with the dissolved metal salt 1, metal ions that are not bonded are removed by dialysis. Subsequently, a metal salt of the type 2 with a smaller redox potential in comparison to metal salt 1 is added in a corresponding excess and the solution is chemically reduced. In this way, clusters of the metal 2 are selectively grown heterogeneously on the DNA molecule wherein the complexes of the metal 1 covalently bonded on the DNA function as nucleation centers. According to this method, different platinum salt complexes are combined with one another so that the reaction kinetics can be controlled within a wide range.

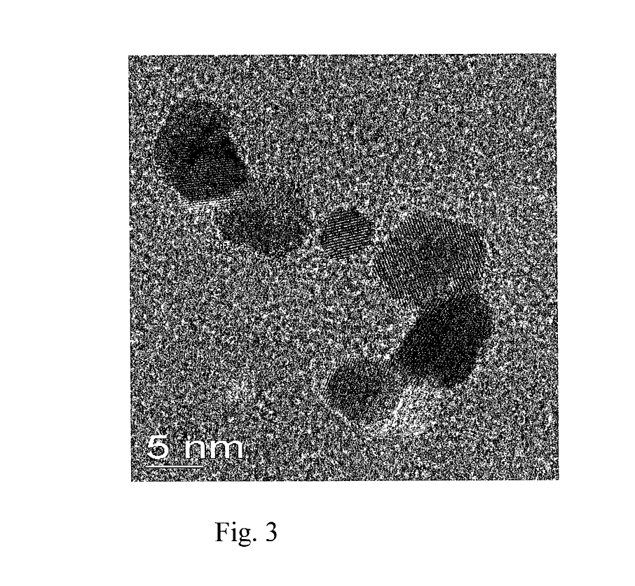

FIG. 3 shows platinum clusters along a DNA strand (high-resolution TEM image, 300 kV). In this example, first metallic nucleation centers were generated with K₂PtCl₄ by means of the described method. In a second step, additional platinum was deposited on the nucleation centers with Pt(NH₃)₂Cl₂ (Cis-platin), and the shown cluster chain resulted.

The incubation of the DNA was realized with K₂PtCl₄ in analogy to the procedure provided in Example 1. After incubation for 20 hours a dialysis relative to water was carried out with a 100 KDa cut-off membrane. The dialysis took 16 hours. In the second step Pt(NH₃)₂Cl₂ was added to the solution (final concentration 1 mM) and immediately reduced with DMAB (final concentration 1 mM). The observed cluster growth kinetics is changed relative to K₂PtCl₄. Note the changed, often hexagonal, morphology of the clusters in comparison to the clusters of FIG. 1.

### EXAMPLE 4

Metal Deposition and Metal Clusters Which Have Been Grown Selectively Heterogeneously on DNA

The procedure described in Example 3 can be slightly modified in order to be able to deposit metals of any type on the pre-fabricated cluster chains which have been formed by selective heterogeneous metal deposition on DNA. For this purpose, after incubation with the dissolved platinum complex metal ions that are not bonded are removed by dialysis. After dialysis, with suitable reduction agents only the platinum complexes bonded to DNA are reduced to metallic platinum in order to initiate growth of ultra-small Pt clusters. This cycle incubation/dialysis/reduction can be repeated, if needed, several times in order to increase the density of the clusters that are present. Subsequently, a metal salt of a second type is added with corresponding excess, and the solution is further reduced. In this way, the metal 2 is selectively deposited on the platinum clusters that are present.

According to this method, gold clusters of a size of approximately 25 nm have been deposited on the DNA in that first nucleation centers are generated on the DNA and, subsequently, a dialysis is performed as described in the embodiment of Example 3. Subsequently, the reduction of the complexes bonded on the DNA by means of DMAB (final concentration 1 mM) is carried out. After reduction, a dialysis is carried out again relative to water for 8 hours. In the second step, clusters grown on the DNA were developed for two minutes with Goldenhance™. As a procedure for this, the standard procedure provided by the manufacturer Nanoprobes, Inc., New York, U.S.A., was used. As an alternative, silver clusters can be generated by reduction of 1 mM AgNO₃ by means of hydrogen.

### EXAMPLE 5

Cluster Nucleation and Cluster Growth with Additional Ligands

An additional stabilization of the platinum clusters by means of sodium citrate was carried out similar to Example 1. In deviation from Example 1, the addition of 0.5 mM trisodium citrate is carried out immediately before addition of the reducing agent.

The reaction kinetics in the presence of citrate is slower. The resulting cluster chains are longer approximately by a factor 3 in comparison to the cluster chains which are produced without addition of sodium citrate. The clusters of the chains generated with sodium citrate are mono-disperse. The clusters are distinctly separated from one another, i.e., they have not grown together.

1. Method for generating nucleation centers for selective heterogeneous growth of metal clusters on DNA molecules, characterized in that a DNA solution is incubated with a metal solution and the entire solution, comprised of metal complex-DNA-adducts and dissolved metal complexes is reduced.

2. Method according to claim 1, characterized in that as a result of the generation of the nucleation centers on the biomolecule the balance between homogeneous and heterogeneous metal cluster nucleation can be affected, by which the metal deposition on the DNA molecules can be controlled.

3. Method according to claim 1, characterized in that a DNA solution is incubated with a platinum salt solution at room temperature and, subsequently, the DNA platinum salt solution is reduced by adding a reducing agent.

4. Method according to claim 3, characterized in that a solution of λ-DNA is incubated with a K₂PtCl₄ solution with a metal complex to nucleotide ratio of approximately 65:1 at room temperature and, subsequently, the DNA metal salt solution is reduced by adding dimethyl amino borane in stoichiometric excess.

5. Method according to claim 1, characterized in that before the reduction excess metal salt is removed by dialysis.

6. Method according to claim 1, characterized in that further protective ligands are added before the reduction.

7. Method according to claim 6, characterized in that sodium citrate is added before the reduction.

8. Method according to claim 1, characterized in that, by incubation of DNA solution with a metal salt solution and subsequent reduction of the resulting metal complex DNA adducts in the presence or absence of dissolved metal complexes, nucleation centers for the selective heterogeneous metal growth are formed selectively or preferred on certain base pairs or base patterns of the DNA.

9. Method for sequence-specific metallization of DNA in which as a function of the sequence, defined portions of the DNA are coated with metals, characterized in that, by incubation of DNA solution with a metal salt solution and subsequent reduction of the resulting metal complex DNA adducts in the presence or absence of dissolved platinum complexes, nucleation centers are formed selectively or preferably on certain base pairs or base patterns of the DNA, which nucleation centers are moreover used for the further metal growth by means of suitable metallization baths.

10. Method according to claim 9, characterized in that a metallization bath comprising a reducing agent and a metal salt solution, identical or different with respect to the incubation and with identical or different metal, is used for metallization.

11. Method for metallization of DNA, characterized in that, by incubation of DNA solution with a metal solution and subsequent reduction of the resulting metal complex DNA adducts in the presence or absence of dissolved metal complexes, nucleation centers are formed and used for the selective heterogeneous metal growth by means of suitable metallization baths.

12. Method according to claim 11, characterized in that a metallization bath comprising a reducing agent and a metal salt solution, identical or different with respect to the incubation and with identical or different metal, is used for metallization.
